(19) United States
(12) Patent Application Publication
(10) Pub. No.: US 2013/0256647 A1
(43) Pub. Date: Oct. 3, 2013

(54) NOVEL ORGANIC COMPOUND AND ORGANIC LIGHT-EMITTING DEVICE INCLUDING SAME

(71) Applicants: Yosuke Nishide, Kawasaki-shi (JP); Naoki Yamada, Inagi-shi (JP); Akihito Saitoh, Gotemba-shi (JP)

(72) Inventors: Yosuke Nishide, Kawasaki-shi (JP); Naoki Yamada, Inagi-shi (JP); Akihito Saitoh, Gotemba-shi (JP)

(73) Assignee: CANON KABUSHIKI KAISHA, Tokyo (JP)

(21) Appl. No.: 13/992,219

(22) PCT Filed: Nov. 10, 2011
(86) PCT No.: PCT/JP2011/076590

§ 371 Date: Jun. 6, 2013

(30) Foreign Application Priority Data
Dec. 9, 2010 (JP) 2010-275135

(51) Int. Cl. H01L 51/00

(52) U.S. Cl. CPC H01L 51/0071; H01L 51/0052; H01L 51/0058
USPC 257/40; 548/126

(57) ABSTRACT

Aspects of the present invention can provide a novel phenanthrothiadiazole compound with the lowest excited triplet level T1 that is high, the phenanthrothiadiazole compound being capable of forming a stable amorphous film. Furthermore, aspects of the present invention can provide an organic light-emitting device having high luminous efficiency and a low driving voltage.

Aspects of the present invention provide a phenanthrothiadiazole compound represented by one of general formulae [1] to [3] according to Claim 1.

### TECHNICAL FIELD

The present invention relates to a novel organic compound and an organic light-emitting device including the novel organic compound.

### BACKGROUND ART

An organic light-emitting device includes a pair of electrodes and an organic compound layer arranged therebetween. The injection of electrons and holes from the respective electrodes produces excitons of a light-emitting compound in the organic compound layer. Light is emitted when the excitons return to the ground state.

Organic light-emitting devices are also referred to as organic electroluminescent devices or organic EL devices.

In an attempt to improve the luminous efficiency of an organic EL device, the use of phosphorescence emission is reported. The luminous efficiency of an organic EL device using phosphorescence emission should be theoretically about four times as high as that of an organic EL device using fluorescence emission.

NPL 1 describes phenanthrothiadiazole-1,1-dioxide (a-1) as an electron-donating unit.

NPL 2 describes a method for synthesizing phenanthrothiadiazole (b-1).

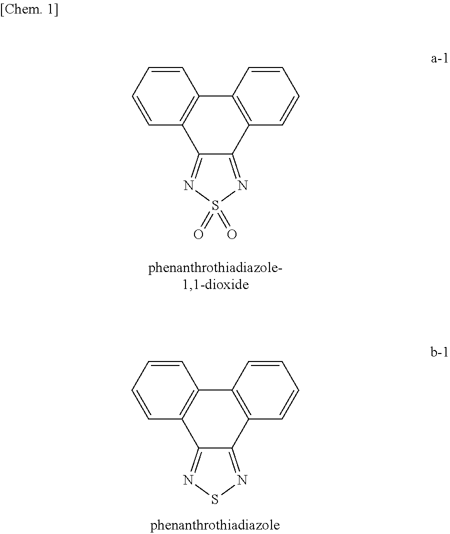 [SMILES: O=S1(=O)N=c2c(c3ccccc3c3ccccc23)=N1 ; c1ccc2c(c1)c1ccccc1c1nsnc21]

### CITATION LIST

### Non Patent Literature

NPL 1 Org. Lett., 2010, 12(20), 4520-4523
NPL 2 J. Org. Chem., 1970, 35(4), 1165-1169

### SUMMARY OF INVENTION

NPL 1 describes phenanthrothiadiazole-1,1-dioxide as an electron-donating unit. However, the lowest excited triplet level T1 of phenanthrothiadiazole-1,1-dioxide is low, so that it is difficult to use phenanthrothiadiazole-1,1-dioxide for a phosphorescence emission device.

NPL 2 describes a method for synthesizing phenanthrothiadiazole. This compound has a high T1 level.

However, phenanthrothiadiazole has a less amorphous nature and thus is not suitably used for organic light-emitting devices.

Aspects of the present invention can provide a novel phenanthrothiadiazole compound having a high T1 level and being capable of forming a stable amorphous film. Furthermore, aspects of the present invention can provide an organic light-emitting device including the novel phenanthrothiadiazole compound, the organic light-emitting device having high luminous efficiency and a low driving voltage.

Accordingly, one disclosed aspect of the present invention provides an organic compound represented by one of general formulae [1] to [3]:

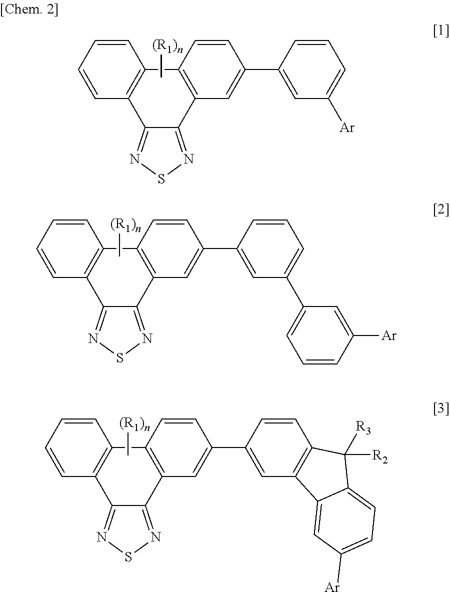

wherein in each of general formulae [1] to [3],

Ar represents a phenyl group, a phenanthryl group, a fluorenyl group, or a triphenylenyl group;

the substituent represented by Ar may be substituted with an alkyl group having 1 to 4 carbon atoms, a phenyl group, a phenanthryl group, a fluorenyl group, or a triphenylenyl group;

R₁ represents an alkyl group having 1 to 4 carbon atoms, n represents an integer of 0 to 3, and when n represents 2 or 3, alkyl groups represented by plural R₁'s may be the same or different; and

R₂ and R₃ each independently represent a hydrogen atom or an alkyl group having 1 to 4 carbon atoms.

Aspects of the present invention provide a new phenanthrothiadiazole compound having a high T1 level and being capable of forming a stable amorphous film. Furthermore, aspects of the present invention provide an organic light-emitting device having high luminous efficiency and a low driving voltage.

### BRIEF DESCRIPTION OF DRAWINGS

FIG. 1 is a schematic cross-sectional view of organic light-emitting devices and switching elements connected to the organic light-emitting devices.

### DESCRIPTION OF EMBODIMENTS

Aspects of the present invention provide an organic compound represented by one of general formulae [1] to [3]:

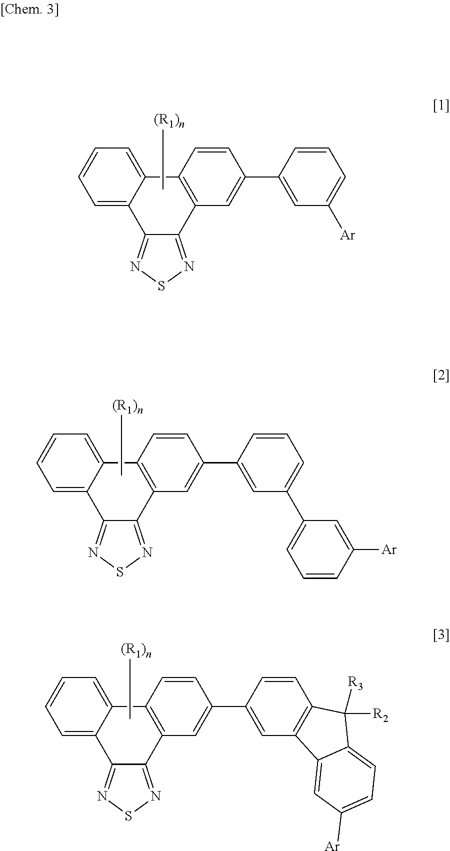

wherein in each of general formulae [1] to [3],

Ar represents a phenyl group, a phenanthryl group, a fluorenyl group, or a triphenylenyl group;

the substituent represented by Ar may be substituted with an alkyl group having 1 to 4 carbon atoms, a phenyl group, a phenanthryl group, a fluorenyl group, or a triphenylenyl group;

R₁ represents an alkyl group having 1 to 4 carbon atoms, n represents an integer of 0 to 3, when n represents 2 or 3, alkyl groups represented by plural R₁'s may be the same or different, and when n represents zero, the phenanthrothiadiazole skeleton is not substituted; and

R₂ and R₃ each independently represent a hydrogen atom or an alkyl group having 1 to 4 carbon atoms.

Comparison of Basic Skeleton (b-1) of Organic Compound According to Aspects of the Present Invention with Compound (a-1) Described in NPL 1

The basic skeleton, phenanthrothiadiazole (b-1), of the organic compound according to aspects of the present invention is compared with phenanthrothiadiazole-1,1-dioxide (a-1) described in NPL 1.

Here, the term “basic skeleton” indicates a fused-ring structure having conjugation.

Phenanthrothiadiazole-1,1-dioxide (a-1), which is a target for comparison, is represented by the following structural formula:

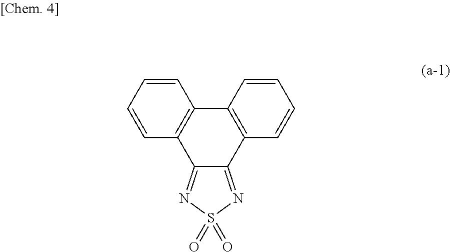 [SMILES: O=S1(=O)N=c2c(c3ccccc3c3ccccc23)=N1]

Phenanthrothiadiazole (b-1), which serves as a basic skeleton of the organic compound according to aspects of the present invention, is represented by the following structural formula:

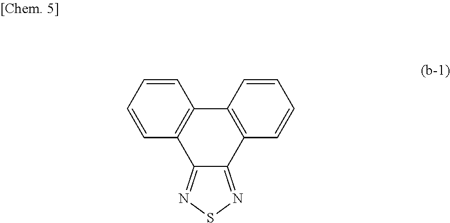 [SMILES: c1ccc2c(c1)c1ccccc1c1nsnc21]

Compound (a-1) and compound (b-1) have different molecular structures and extremely different properties and thus are different skeletons. The sulfur atom of compound (b-1) of the organic compound according to aspects of the present invention has a formal oxidation number of +2 and two lone pairs. One of the lone pairs is used for π conjugation. Thus, the skeleton (b-1) satisfies the Hückel rule and exhibits aromaticity. On the other hand, the sulfur atom of compound a-1, which is a comparative compound, has a formal oxidation number of +6 and no lone pair. Thus, the skeleton (a-1) does not satisfy the Hückel rule or aromaticity. As described above, the basic skeleton (b-1) of the organic compound having aromaticity according to aspects of the present invention is different from the skeleton of comparative compound (a-1) that does not have aromaticity.

Furthermore, for example, T1 levels differ greatly between the skeletons. Phenanthrothiadiazole-1,1-dioxide (a-1), which is a comparative compound, has a low T1 level. So, a compound having a basic skeleton of phenanthrothiadiazole-1,1-dioxide is not suitable as a material for use in a green phosphorescent light-emitting device.

Meanwhile, phenanthrothiadiazole (b-1), which serves as a basic skeleton of the organic compound according to aspects of the present invention, has a high T1 level. So, a compound having the basic skeleton is suitably used as a basic skeleton of a material for use in a green phosphorescent light-emitting device.

Table 1 shows the calculated values and measured values in toluene solutions (at 77 K) of T1 levels of compounds (a-1) and (b-1). The calculations were performed using molecular orbital calculations described below. The wavelengths of rising edges in spectra of compounds (a-1) and (b-1) were defined as the measured values of the T1 levels.

As is apparent from the results shown in Table 1, a compound having a basic skeleton of compound (a-1), which is a comparative compound, is not suitable as a material for use in a green phosphorescent light-emitting device because of its low T1 level. Meanwhile, the organic compound having a basic skeleton of phenanthrothiadiazole (b-1) according to aspects of the present invention has a high T1 level and thus can emit light with high efficiency when used in an organic layer of a green phosphorescent light-emitting device.

TABLE 1

| Compound | a-1 | b-1 |
|---|---|---|
| Structural formula | 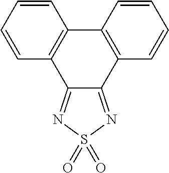 [SMILES: O=S1(=O)N=c2c(c3ccccc3c3ccccc23)=N1] | 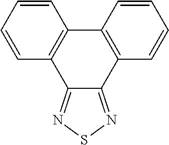 [SMILES: c1ccc2c(c1)c1ccccc1c1nsnc21] |
| T1 (nm) | 627 | 450 |
| *calulated | | |
| value | | |
| T1 (nm) | 627 | 445 |
| *measured | | |
| value | | |

T1, HOMO, and LUMO were determined by molecular orbital calculations as described below.

The molecular orbital calculations were performed by widely-used Gaussian 03 (Gaussian 03, Revision D. 01, M. J. Frisch, G. W. Trucks, H. B. Schlegel, G. E. Scuseria, M. A. Robb, J. R. Cheeseman, J. A. Montgomery, Jr., T. Vreven, K. N. Kudin, J. C. Burant, J. M. Millam, S. S. Iyengar, J. Tomasi, V. Barone, B. Mennucci, M. Cossi, G. Scalmani, N. Rega, G. A. Petersson, H. Nakatsuji, M. Hada, M. Ehara, K. Toyota, R. Fukuda, J. Hasegawa, M. Ishida, T. Nakajima, Y. Honda, O. Kitao, H. Nakai, M. Klene, X. Li, J. E. Knox, H. P. Hratchian, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, P. Y. Ayala, K. Morokuma, G. A. Voth, P. Salvador, J. J. Dannenberg, V. G. Zakrzewski, S. Dapprich, A. D. Daniels, M. C. Strain, O. Farkas, D. K. Malick, A. D. Rabuck, K. Raghavachari, J. B. Foresman, J. V. Ortiz, Q. Cui, A. G. Baboul, S. Clifford, J. Cioslowski, B. B. Stefanov, G. Liu, A. Liashenko, P. Piskorz, I. Komaromi, R. L. Martin, D. J. Fox, T. Keith, M. A. Al-Laham, C. Y. Peng, A. Nanayakkara, M. Challacombe, P. M. W. Gill, B. Johnson, W. Chen, M. W. Wong, C. Gonzalez, and J. A. Pople, Gaussian, Inc., Wallingford Conn., 2004) by means of the density functional theory (DFT) using the 6-31+G(d) basis set.

The organic compound according to aspects of the present invention has a T1 level suitable for a green phosphorescent light-emitting device.

Phenanthrothiadiazole, which is the basic skeleton of the organic compound according to aspects of the present invention, has a high T1 level as shown in Table 1.

The organic compound according to aspects of the present invention has a structure in which phenanthrothiadiazole, which serves as a basic skeleton, is substituted with a substituent and maintains the high T1 level of the basic skeleton.

To maintain the high T1 level of the basic skeleton, it is necessary to use a substituent having a high T1 level.

Furthermore, in order not to extend the conjugation more than necessary to reduce the T1 level, a linking group or the position of the bond between a linking group and a substituent needs to be selected.

Thus, the organic compound according to aspects of the present invention can be substituted with, for example, a phenyl group, a phenanthryl group, a triphenylene group, or a fluorenyl group, which is a substituent having a high T1 level. In addition, linking groups as illustrated below may be used. That is, the use of a m-phenylene group, m-biphenylene group, or a 3,6-fluorenylene group suppresses the extension of conjugation to provide a compound having a high T1 level.

At the para position, the conjugation extends, thus causing difficulty in maintaining a high T1 level.

Consequently, the use of the organic compound according to aspects of the present invention for a green phosphorescent light-emitting device results in high-efficient light emission.

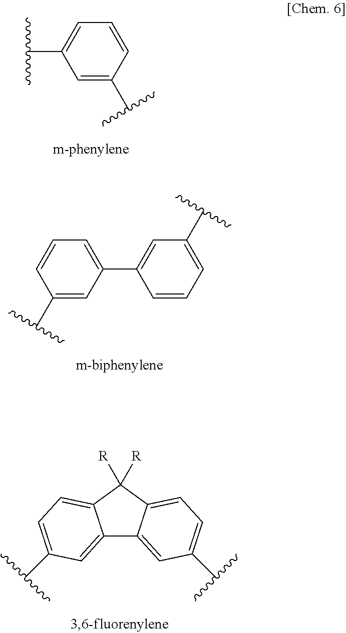

It is difficult to form an amorphous film composed of phenanthrothiadiazole (b-1) described in NPL 2. So, phenanthrothiadiazole (b-1) is not suitable as a material for use in an organic light-emitting device.

Meanwhile, the organic compound according to aspects of the present invention has a structure including phenanthrothiadiazole serving as a basic skeleton, the substituent, and the linking group as represented by one of general formulae [1] to [3] and thus is capable of forming a stable amorphous film.

As described above, the organic compound including the linking group and the substituent according to aspects of the present invention has a high T1 level and high amorphous nature. A compound having high amorphous nature is suitable for an organic light-emitting device.

The organic compound according to aspects of the present invention has the phenanthrothiadiazole skeleton and thus has a deep level of the lowest unoccupied molecular orbital (LUMO) and an excellent capability of transporting electrons. The expression “deep level of the LUMO” indicates that the LUMO level is farther from the vacuum level.

The organic compound according to aspects of the present invention can be used as a material for use in a green phosphorescent light-emitting device.

In this embodiment, the T1 level suitable for a green phosphorescent light-emitting device is 490 nm or less in terms of the phosphorescence emission wavelength.

In this embodiment, the wavelength of green light emitted is defined in the range of 490 nm to 530 nm.

So, a material used as a host for a hole transport layer, an exciton-blocking layer, an electron transport layer, and a light-emitting layer in a green phosphorescent light-emitting device according to this embodiment can phosphoresce at 490 nm or less.

In the case where a material that phosphoresces at a shorter wavelength, i.e., at a higher energy level, than that of a light-emitting material is used around the light-emitting layer, the energy transfer to a material other than a dopant is suppressed, thereby resulting in highly efficient emission from the dopant.

In the case where the organic compound according to aspects of the present invention is used as, in particular, an exciton-blocking material, an electron injection (transport) material, and a host material for use in an organic light-emitting device, it is possible to reduce the driving voltage and increase the efficiency.

This is because the phenanthrothiadiazole skeleton has an electron-withdrawing structure with a deep LUMO level and easily receives electrons compared with phenanthrene, triphenylene, and so forth.

The reason for the reduction in driving voltage is that the deep LUMO level results in low energy barriers between a cathode, the electron injection (transport) layer, and the exciton-blocking layer to facilitate electron injection.

In the case where the organic compound according to aspects of the present invention is used as, in particular, an exciton-blocking material, an electron injection (transport) material, and a host material for use in a green phosphorescent light-emitting device, the electron injection is facilitated, thus reducing the driving voltage and increasing the efficiency.

Accordingly, in the case where the organic compound according to aspects of the present invention is used for an organic light-emitting device, the resulting organic light-emitting device has high stability and long life.

### Exemplification of Organic Compound According to Aspects of the Present Invention

Non-limiting examples of the compounds represented by general formulae [1] to [3] are described below.

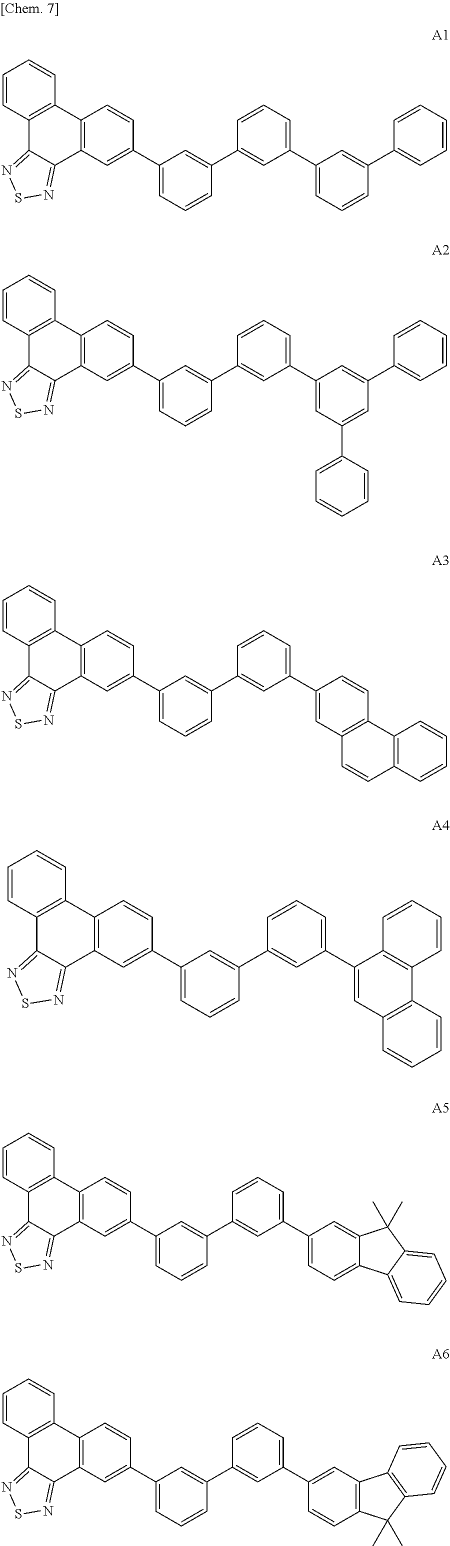

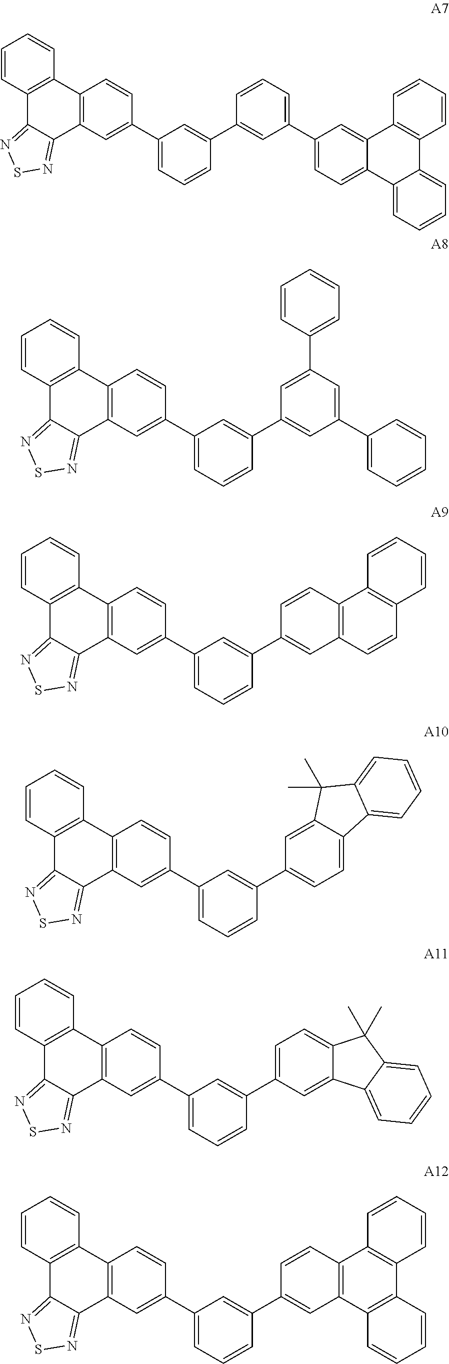

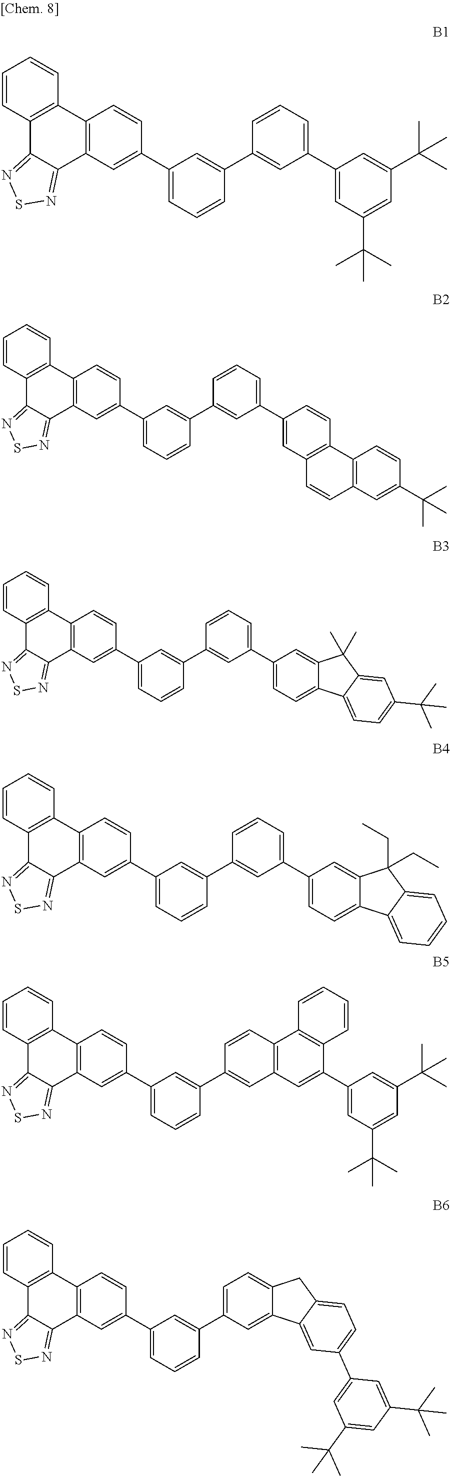

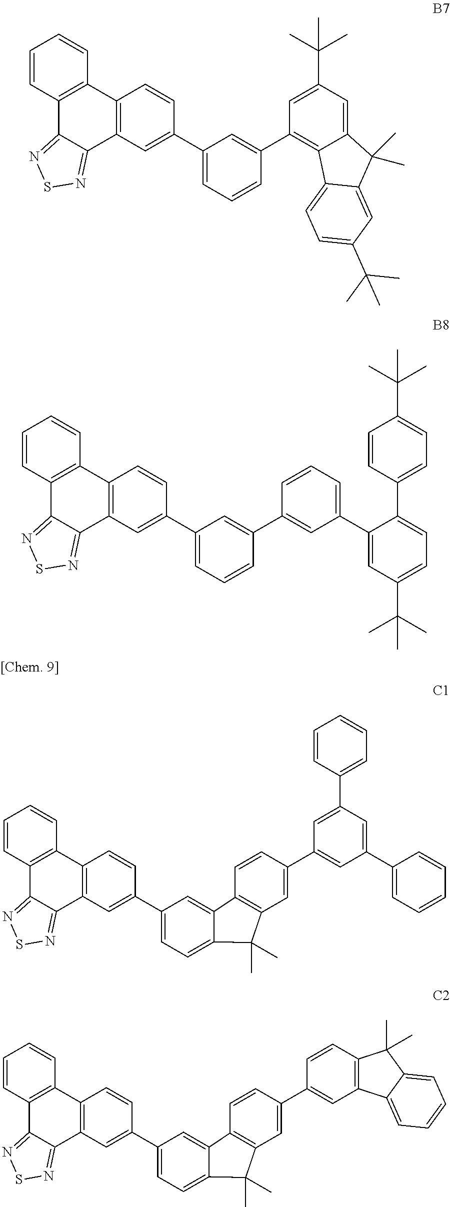

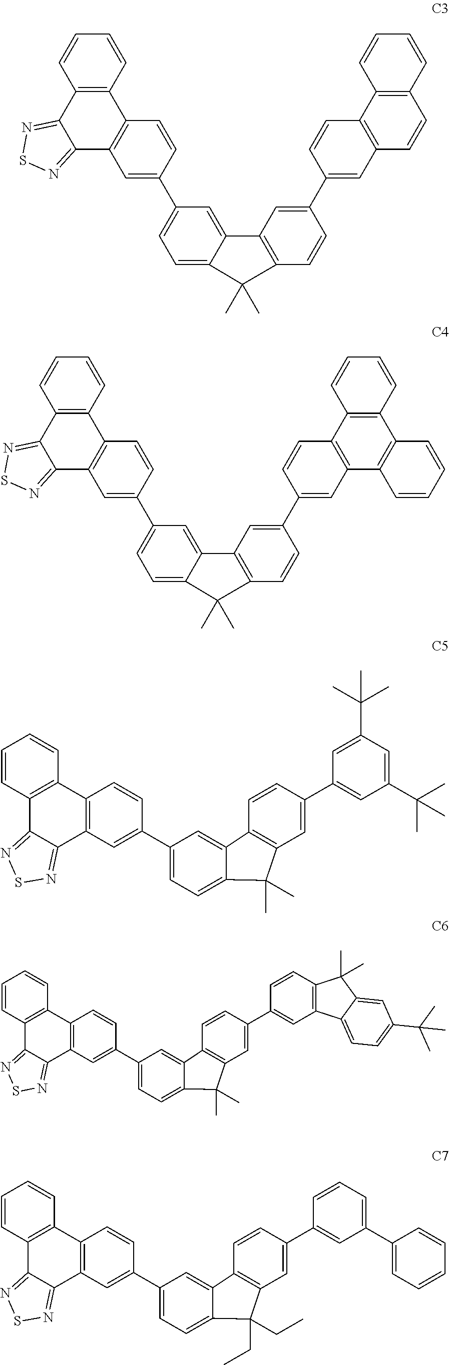

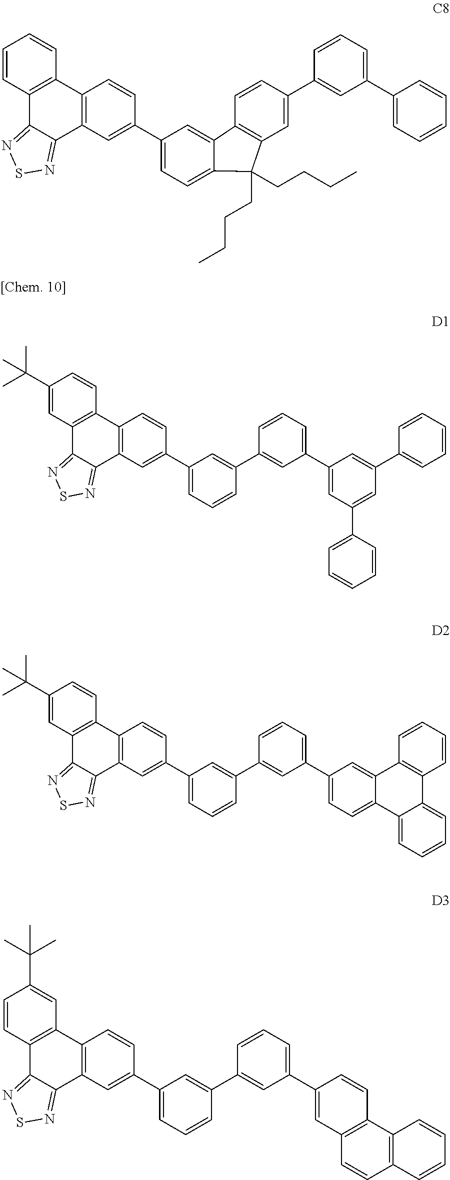

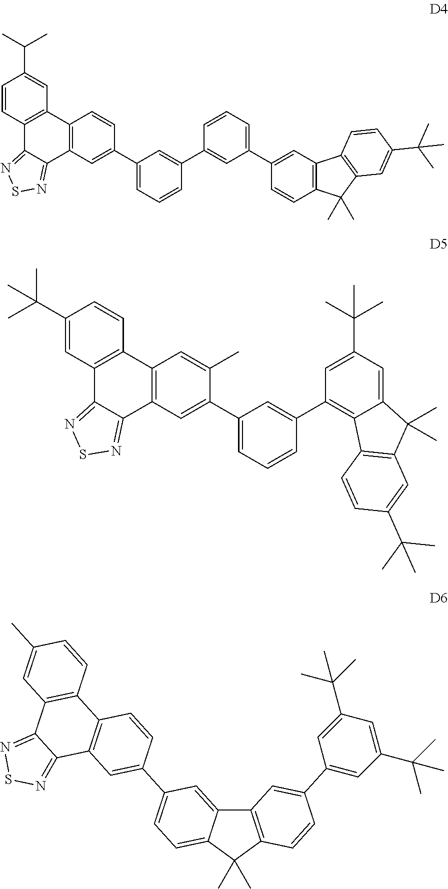

### Properties of Exemplified Compounds

The compounds represented by general formulae [1] and [2] are categorized as compound group A and compound group B. The compounds represented by general formula [3] are categorized as compound group C. Each of the linking groups serves to interrupt the conjugation between the phenanthrothiadiazole skeleton and the Ar moiety illustrated in the general formula. This results in the compounds having high T1 levels in the wavelength range shorter than 490 nm.

The aryl groups expressed as Ar's in general formulae [1] to [3] are selected from aryl groups such that the compounds represented by general formulae [1] to [3] have T1 levels in the wavelength range shorter than 490 nm. Specifically, the aryl groups include a phenyl group, a phenanthryl group, a fluorenyl group, and a triphenyl group.

These groups may have substituents such that their T1 levels are in the wavelength range shorter than 490 nm. Specifically, these groups include an alkyl group having 1 to 4 carbon atoms, a phenyl group, a phenanthryl group, a fluorenyl group, and a triphenylenyl group.

Table 2 shows the calculated values of T1 levels of the organic compounds according to aspects of the present invention. The calculations were performed as in Table 1. The measured values were values measured in toluene solutions at 77 K. The wavelengths of rising edges in spectra of the compounds were defined as the measured values of the T1 levels in Table 2.

The results demonstrate that the compounds, which are represented by general formulae [1] to [3], substituted with a phenyl group, a phenanthryl group, a fluorenyl group, and a triphenyl group serving as Ar's have T1 levels in the wavelength range shorter than 490 nm. Substantially the same T1 values are obtained regardless of which substituent is used. This is because the linking groups and the aryl groups attached to the phenanthrothiadiazole skeleton have higher T1 levels than phenanthrothiadiazole skeleton.

TABLE 2

| Exemplified | | |
|---|---|---|
| compound | T1 nm (calculated value) | T1 nm (measured value) |
| A-1 | 469 | 469 |
| A-3 | 470 | 471 |
| A-5 | 470 | 470 |
| A-7 | 469 | 470 |

Compound group A and C1 to C4 in compound group C have high-planarity aryl substituents as Ar's in general formulae [1] to [3], as compared with compound group B.

So, these compounds have higher degrees of intermolecular stacking than compound group B in the form of thin films and have high mobility of holes and electrons. Among compound groups A and C, in particular, compounds A3, A5 to A7, A9 to 12, and C2 to 4, which have aryl groups selected from fluorenyl, phenanthryl, and triphenylene groups, have high electron mobility.

The reason for this is that the high electron mobility of the compounds reflects the high electron mobility of the fluorene skeleton, the phenanthrene skeleton, and the triphenylene skeleton.

Each of the compounds illustrated in compound groups A and B has a m-phenylene or m-biphenylene linking group and thus many rotatable portions in its molecule, thereby advantageously resulting in its low sublimation temperature and a low evaporation temperature at the time of the production of an organic light-emitting device.

The compounds illustrated in compound group C are characterized by having high glass-transition temperatures due to the presence of 3,6-fluorenylene linking groups, as compared with compounds each having a m-phenylene or m-biphenylene linking group. This is because the high rigidity of these molecules suppresses molecular motion.

The compounds illustrated in compound group B and compounds C5 and C6 in compound group C have bulky aryl substituents serving as Ar's.

Specifically, the compounds have aryl groups each substituted with an alkyl group having 1 to 4 carbon atoms. These compounds are sterically bulky, thus suppressing intermolecular stacking and concentration quenching.

In addition, the compounds have low degrees of intermolecular stacking and the low mobility of holes and electrons, as compared with the compounds illustrated in compound group A and C1 to C4 in compound group C.

The compounds illustrated in compound group D each have phenanthrothiadiazole, which serves as a basic skeleton, substituted with an alkyl group having 1 to 4 carbon atoms.

The calculation results shown in Table 3 demonstrate that even if phenanthrothiadiazole, which serves as a basic skeleton, is substituted with an alkyl group, the high T1 level is maintained.

The LUMO distribution of the organic compound according to aspects of the present invention was determined by calculations and found to be localized around phenanthrothiadiazole, which serves as a basic skeleton. In a compound having an alkyl group-substituted phenanthrothiadiazole skeleton, intermolecular stacking can be suppressed. Thus, the energy levels of the HOMO and LUMO can be finely adjusted by the selection of the type and number of alkyl groups.

TABLE 3

| Compound | b-1 | b-2 |
|---|---|---|
| Structural formula | 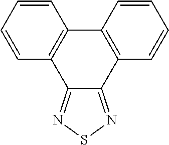 [SMILES: c1ccc2c(c1)c1ccccc1c1nsnc21] | 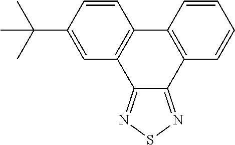 [SMILES: CC(C)(C)c1ccc2c3ccccc3c3nsnc3c2c1] |
| T1 (nm) | 450 | 453 |
| *calculated value | | |

In particular, the organic compound according to aspects of the present invention can be represented by general formula [4]:

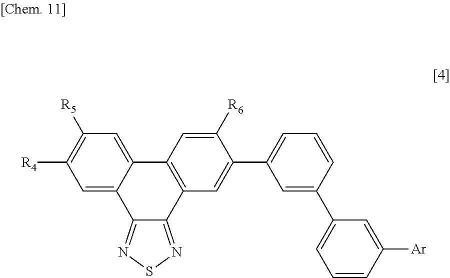

wherein in general formula [4], R₄ to R₆ each independently represent a hydrogen atom or an alkyl group having 1 to 4 carbon atoms;

Ar represents a phenyl group, a phenanthryl group, a fluorenyl group, or a triphenylenyl group; and

the substituent represented by Ar may be substituted with an alkyl group having 1 to 4 carbon atoms, a phenyl group, a phenanthryl group, a fluorenyl group, or a triphenylenyl group.

The organic compound according to aspects of the present invention may be used for not only an exciton-blocking layer but also a light-emitting layer, an electron injection (transport) layer, and so forth of an organic light-emitting device.

Furthermore, the organic compound according to aspects of the present invention may be used for not only a green phosphorescent light-emitting device but also a red phosphorescence light-emitting device. In this case, the organic compound can be used for an exciton-blocking layer, a light-emitting layer, an electron injection (transport) layer, and so forth of an organic light-emitting device.

Moreover, in the case where the organic compound is used for the exciton-blocking layer and the electron injection (transport) layer, the organic compound can be used for an organic light-emitting device, such as a phosphorescent light-emitting device or a fluorescent light-emitting device, which emits any colored light. For example, the organic compound may be used for a blue-light-emitting device, a blue-green-light-emitting device, a light-blue-light-emitting device, a green-light-emitting device, a yellow-light-emitting device, an orange-light-emitting device, a red-light-emitting device, and a white-light-emitting device.

### Explanation of Synthetic Route

An exemplary synthetic route for an organic compound according to aspects of the present invention will be described. Reaction schemes are illustrated below.

Intermediate E2 may be prepared by, for example, allowing E1 to react with triethylamine and thionyl chloride in a dichloromethane solvent.

Intermediate E5 may be prepared by, for example, allowing E2 to react with NBS in a dichloromethane solvent in the presence of trifluoromethanesulfonic acid as a catalyst.

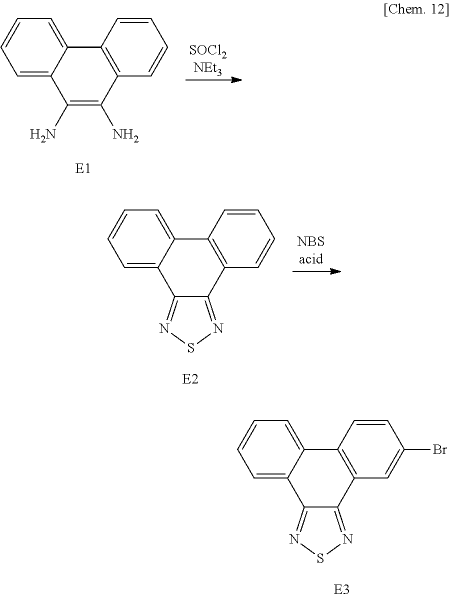 [SMILES: Nc1c(N)c2ccccc2c2ccccc12 ; c1ccc2c(c1)c1ccccc1c1nsnc21 ; Brc1ccc2c3ccccc3c3nsnc3c2c1]

An organic compound according to aspects of the present invention may be prepared by, for example, allowing E3 to react with E4 (boronic acid or pinacolborane) in a toluene-ethanol-distilled water mixed solvent in the presence of sodium carbonate and Pd(PPh₃)₄ as a catalyst.

The use of different compounds as E4 may provide various organic compounds. Table 4 shows specific examples of synthetic compounds. Similarly, the use of compounds substituted with alkyl groups in place of E3 may prepare the exemplified compounds in compound group D.

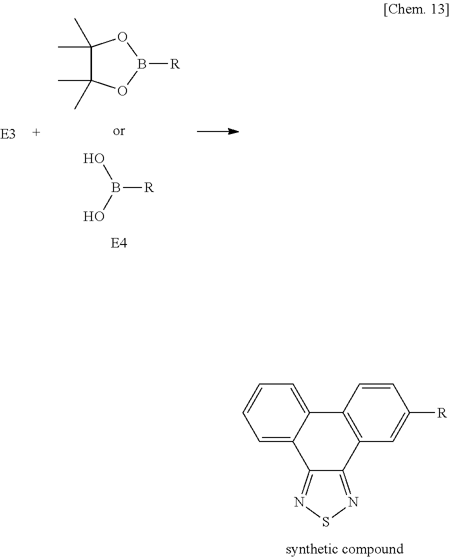

TABLE 4

| | | | Ex- |
|---|---|---|---|
| | | | empli- |
| | | | fied |
| | | | com- |
| | Intermediate D9 | Synthetic compound | pound |
| 1 | 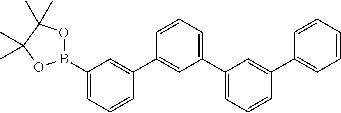 [SMILES: CC1(C)OB(c2cccc(-c3cccc(-c4cccc(-c5ccccc5)c4)c3)c2)OC1(C)C] | 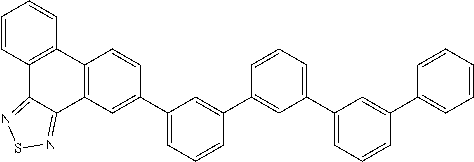 [SMILES: c1ccc(-c2cccc(-c3cccc(-c4cccc(-c5ccc6c7ccccc7c7nsnc7c6c5)c4)c3)c2)cc1] | A1 |
| 2 | 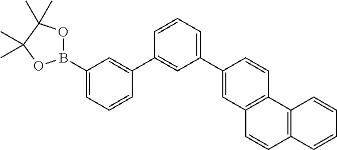 [SMILES: CC1(C)OB(c2cccc(-c3cccc(-c4ccc5c(ccc6ccccc65)c4)c3)c2)OC1(C)C] | 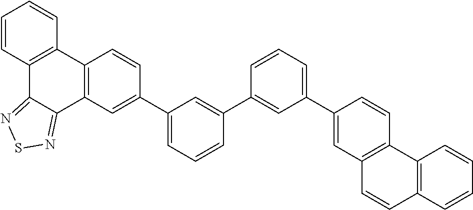 [SMILES: c1cc(-c2cccc(-c3ccc4c5ccccc5c5nsnc5c4c3)c2)cc(-c2ccc3c(ccc4ccccc43)c2)c1] | A3 |
| 3 | 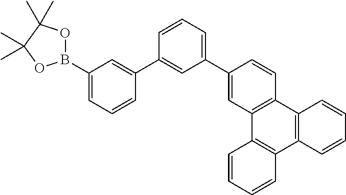 [SMILES: CC1(C)OB(c2cccc(-c3cccc(-c4ccc5c6ccccc6c6ccccc6c5c4)c3)c2)OC1(C)C] | 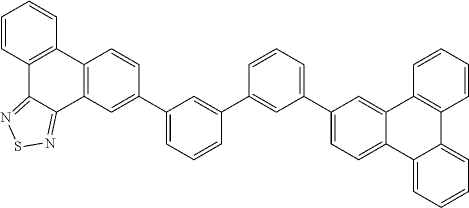 [SMILES: c1cc(-c2cccc(-c3ccc4c5ccccc5c5nsnc5c4c3)c2)cc(-c2ccc3c4ccccc4c4ccccc4c3c2)c1] | A7 |
| 4 | 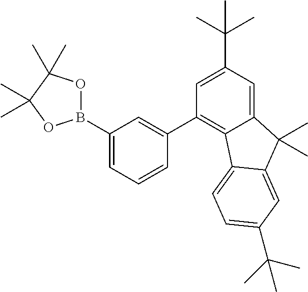 | 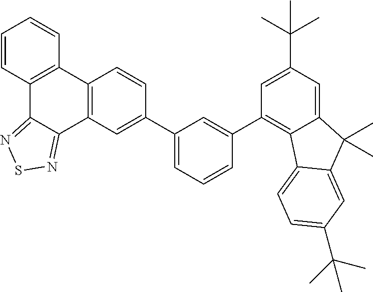 [SMILES: CC(C)(C)c1ccc2c(c1)C(C)(C)c1cc(C(C)(C)C)cc(-c3cccc(-c4ccc5c6ccccc6c6nsnc6c5c4)c3)c1-2] | B7 |
| 5 | 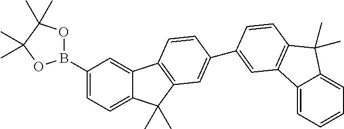 [SMILES: CC1(C)c2ccccc2-c2cc(-c3ccc4c(c3)C(C)(C)c3ccc(B5OC(C)(C)C(C)(C)O5)cc3-4)ccc21] | 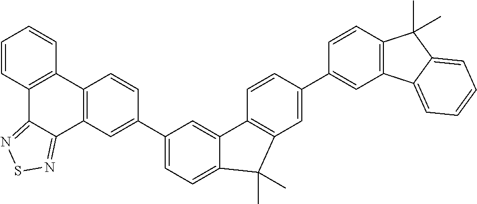 [SMILES: CC1(C)c2ccccc2-c2cc(-c3ccc4c(c3)C(C)(C)c3ccc(-c5ccc6c7ccccc7c7nsnc7c6c5)cc3-4)ccc21] | C2 |
| 6 | 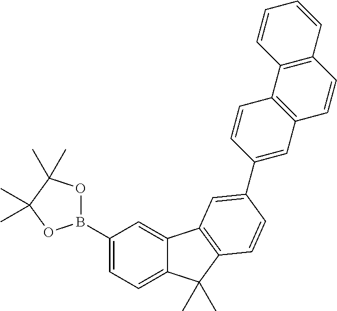 [SMILES: CC1(C)c2ccc(B3OC(C)(C)C(C)(C)O3)cc2-c2cc(-c3ccc4c(ccc5ccccc54)c3)ccc21] | 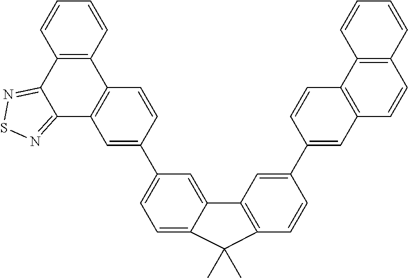 [SMILES: CC1(C)c2ccc(-c3ccc4c(ccc5ccccc54)c3)cc2-c2cc(-c3ccc4c5ccccc5c5nsnc5c4c3)ccc21] | C3 |
| 7 | 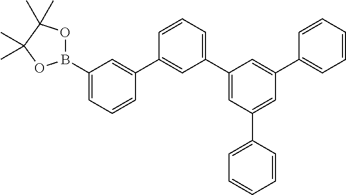 [SMILES: CC1(C)OB(c2cccc(-c3cccc(-c4cc(-c5ccccc5)cc(-c5ccccc5)c4)c3)c2)OC1(C)C] | 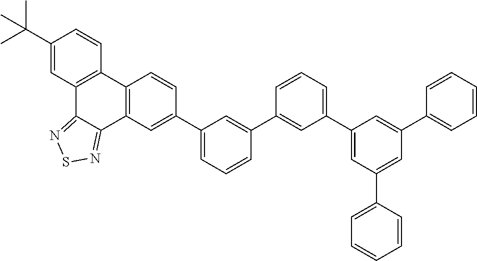 [SMILES: CC(C)(C)c1ccc2c3ccc(-c4cccc(-c5cccc(-c6cc(-c7ccccc7)cc(-c7ccccc7)c6)c5)c4)cc3c3nsnc3c2c1] | D1 |

### Explanation of Organic Light-Emitting Device

An organic light-emitting device according to this embodiment will be described below.

The organic light-emitting device according to this embodiment includes an organic compound layer provided between an anode and a cathode, which are a pair of electrodes. The organic compound layer contains the organic compound represented by one of general formulae [1] to [4].

The organic compound layer of the organic light-emitting device according to aspects of the present invention may have a single-layer structure or a multilayer structure. The multilayer structure includes a plurality of layers appropriately selected from, for example, a hole injection layer, a hole transport layer, a light-emitting layer, a hole-blocking layer, an electron transport layer, an electron injection layer, and an exciton-blocking layer. Of course, plural layers may be selected from the foregoing layers and used in combination.

The structure of the organic light-emitting device according to this embodiment is not limited thereto. Various layer structures may be used. Examples of the layer structures include a structure in which an insulating layer is arranged at the interface between an electrode and the organic compound layer; a structure in which a adhesive layer or an interference layer is arranged; and a structure in which an electron transport layer or a hole transport layer includes two sublayers having different ionization potentials.

The organic light-emitting device according to aspects of the present invention may have a bottom-emission structure in which light emerges from an electrode adjacent to a substrate, a top-emission structure in which light emerges from a surface opposite a substrate, or a structure in which light emerges from both surfaces.

The organic compound represented by one of general formulae [1] to [3] according to aspects of the present invention can be used for an exciton-blocking layer. This is because the organic compound according to aspects of the present invention has a high T1 level and thus can suppress the leakage of excitons generated in a light-emitting layer.

While the organic compound is particularly effective in a green phosphorescent light-emitting device, the organic compound may be used for other organic light-emitting devices without limitation.

Phenanthrothiadiazole, which serves as a basic skeleton of the organic compound according to aspects of the present invention, is characterized by having an electron-withdrawing structure, a deep LUMO level, and an excellent capability of transporting electrons.

So, the organic compound represented by one of general formulae [1] to [3] according to aspects of the present invention may be used for an electron injection (transport) layer. The electron injection (transport) layer may be doped with an alkali metal, e.g., lithium or cesium, an alkaline-earth metal, e.g., calcium, or a salt thereof.

The use of the exciton-blocking layer or the electron injection (transport) layer composed of the organic compound according to this embodiment can provide an organic light-emitting device that can be driven at a low voltage.

The organic compound according to aspects of the present invention may be used as a host material or a guest material in a light-emitting layer. Furthermore, the organic compound may be used as an assist material.

Here, the term “host material” indicates a compound whose proportion by weight is the highest in the light-emitting layer. The term “guest material” indicates a compound whose proportion by weight is lower than that of the host material in the light-emitting layer and which is mainly responsible for light emission. The assist material or a second host material is defined as a compound whose proportion by weight is lower than that of the host material in the light-emitting layer and which assists in the emission of light from the guest material.

In particular, in the case where the organic compound is used as a phosphorescent host material and combined with a guest material which emits light in the green-to-red region and which has an emission peak in the range of 490 nm to 660 nm, the loss of the triplet energy is low, thus increasing the efficiency of the light-emitting device.

In the organic light-emitting device according to this embodiment, the light-emitting layer has a host material content of 50% by weight to 99.9% by weight and preferably 80% by weight to 99.9% by weight with respect to the total weight of the light-emitting layer.

In the case where the organic compound according to this embodiment is used as a guest material, the guest material content is preferably in the range of 0.1% by weight to 30% by weight and more preferably 0.5% by weight to 10% by weight with respect to the host material.

The organic light-emitting device according to this embodiment may contain a known material, for example, a low- or high-molecular weight hole injection material, hole transport material, host material, guest material, electron injection material, or electron transport material, together with the organic compound according to aspects of the present invention, as needed.

Examples of these compounds are described below.

As the hole injection material or hole transport material, a material having a high hole mobility can be used. Examples of low- and high-molecular weight materials having the capability of injecting or transporting holes include, but are not limited to, triarylamine derivatives, phenylenediamine derivatives, stilbene derivatives, phthalocyanine derivatives, porphyrin derivatives, poly(vinyl carbazole), polythiophene, and other electrically conductive polymers.

Examples of the host material include, but are not limited to, triarylamine derivatives, phenylene derivatives, fused-ring aromatic compounds, such as naphthalene derivatives, phenanthrene derivatives, fluorene derivatives, and chrysene derivatives, organometallic complexes, such as organoaluminum complexes, e.g., tris(8-quinolinolato)aluminum, organoberyllium complexes, organoiridium complexes, and organoplatinum complexes, and polymer derivatives, such as poly(phenylene vinylene) derivatives, polyfluorene derivatives, polyphenylene derivatives, poly(thienylene vinylene) derivatives, and polyacetylene derivatives.

Examples of the guest material include, but are not limited to, phosphorescent Ir complexes described below and platinum complexes.

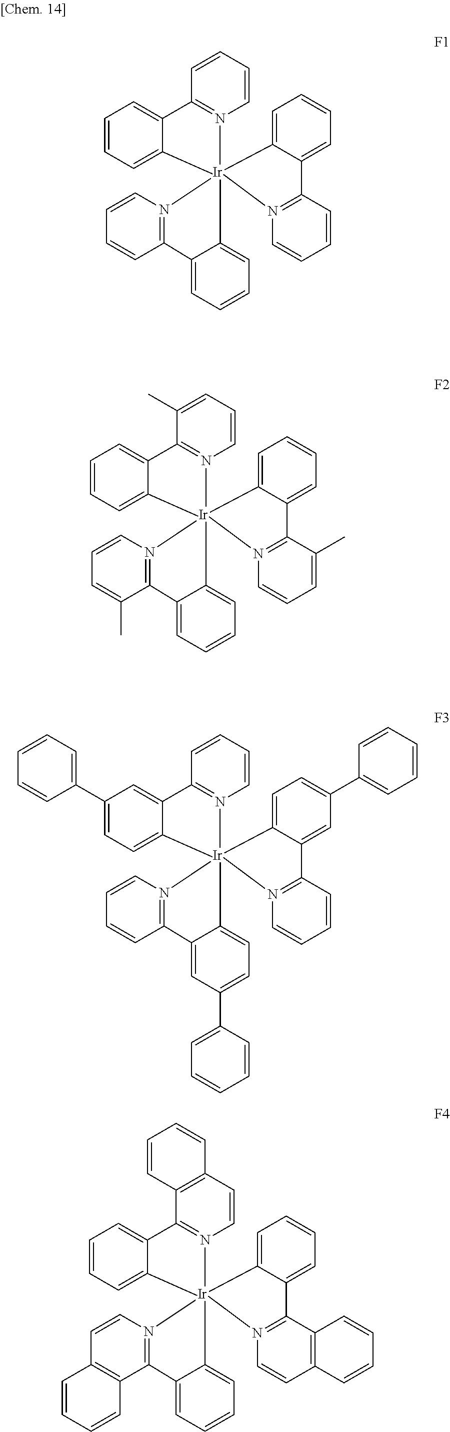

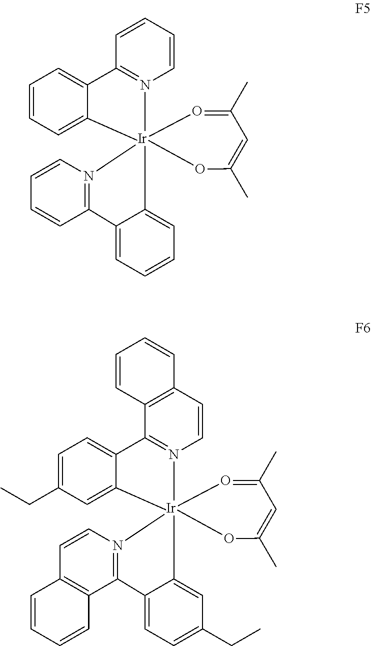

A fluorescent dopant may also be used. Examples thereof include fused-ring compounds, such as fluorene derivatives, naphthalene derivatives, pyrene derivatives, perylene derivatives, tetracene derivatives, anthracene derivatives, and rubrene, quinacridone derivatives, coumarin derivatives, stilbene derivatives, organoaluminum complexes, such as tris(8-quinolinolato)aluminum, organoberyllium complexes, and polymer derivatives, such as poly(phenylene vinylene) derivatives, polyfluorene derivatives, and polyphenylene derivatives.

The electron injection material or electron transport material is selected in view of, for example, the hole mobility of the hole injection material or hole transport material. Examples of the electron injection material or electron transport material include, but are not limited to, oxadiazole derivatives, oxazole derivatives, pyrazine derivatives, triazole derivatives, triazine derivatives, quinoline derivatives, quinoxaline derivatives, phenanthroline derivatives, and organoaluminum complexes.

As a material for an anode, a material having a higher work function can be used. Examples of the material that can be used include elemental metals, such as gold, platinum, silver, copper, nickel, palladium, cobalt, selenium, vanadium, and tungsten, and alloys thereof; and metal oxides, such as tin oxide, zinc oxide, indium oxide, indium tin oxide (ITO), and indium zinc oxide. Furthermore, conductive polymers, such as polyaniline, polypyrrole, and polythiophene, may be used. These materials for the electrode may be used alone or in combination. The anode may have a single-layer structure or multilayer structure.

As a material for a cathode, a material having a lower work function can be used. Examples of the material include elemental metals, such as alkali metals, e.g., lithium and cesium, alkaline-earth meals, e.g., calcium, and aluminum, titanium, manganese, silver, lead, and chromium, and alloys thereof. Examples of the alloys that can be used include magnesium-silver, aluminum-lithium, and aluminum-magnesium. Metal oxides, such as indium tin oxide (ITO), may be used. These materials for the electrode may be used alone or in combination. The cathode may have a single-layer structure or multilayer structure.

A layer included in the organic light-emitting device according to this embodiment is formed by a method described below.

Typically, the layer may be formed by a vacuum evaporation method, an ionized evaporation method, a sputtering method, or a method using plasma. Alternatively, the layer may be formed by a known coating method, e.g., spin coating, dipping, a casting method, the Langmuir-Blodgett (LB) technique, or an ink-jet method, using a solution of a material dissolved in an appropriate solvent. Here, the formation of the layer by, for example, the vacuum evaporation method or the coating method, is less likely to cause crystallization or the like, resulting in excellent temporal stability. Furthermore, in the case of forming the layer by the coating method, the layer may be formed in combination with an appropriate binder resin.

Examples of the binder resin include, but are not limited to, polyvinylcarbazole resins, polycarbonate resins, polyester resins, acrylonitrile-butadiene-styrene (ABS) resins, acrylic resins, polyimide resins, phenolic resins, epoxy resins, silicone resins, and urea resins. These binder resins may be used alone in the form of a homopolymer or copolymer. Alternatively, these binder resins may be used in combination as a mixture. In addition, known additives, such as a plasticizer, an antioxidant, and an ultraviolet absorber, may be used in combination, as needed.

### Application of Organic Light-Emitting Device

The organic light-emitting device according to aspects of the present invention may be used for display apparatuses, illuminating apparatuses, exposure light sources for use in electrophotographic image forming apparatuses, and backlights for use in liquid crystal displays.

A display apparatus includes the organic light-emitting device according to aspects of the present invention in a display unit. The display unit includes a plurality of pixels. Each of the pixels includes the organic light-emitting device according to this embodiment and a TFT element, which is an exemplary switching element, configured to control luminance. A drain electrode or source electrode is connected to an anode or cathode of the organic light-emitting device. The display apparatus may be used as an image display apparatus for personal computers and so forth.

The display apparatus may be an image input apparatus that includes an image input unit configured to input image information from, for example, an area CCD sensor, a linear CCD sensor, or a memory card and to output the image information to a display unit. Furthermore, the display apparatus may have both functions: as a display unit included in an image pick-up apparatus or an ink-jet printer, an image output function that displays image information supplied from the outside; and as an operation panel, an input function that inputs processing information to an image. In addition, the display apparatus may be used for a display unit of a multifunction printer.

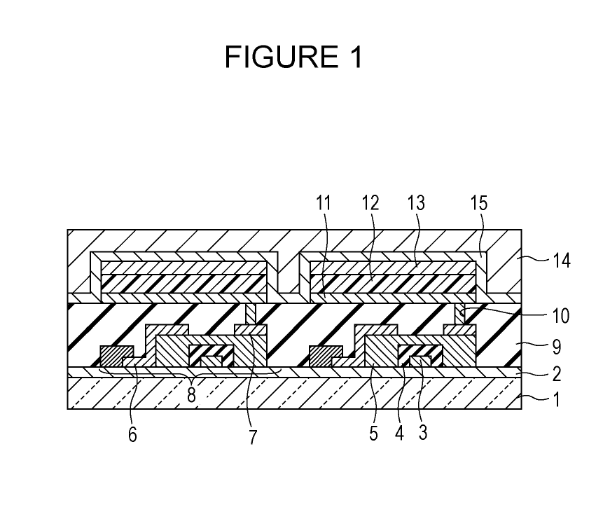

A display apparatus including the organic light-emitting device according to this embodiment will be described below with reference to FIG. 1.

FIG. 1 is a schematic cross-sectional view of a display apparatus including organic light-emitting devices according to this embodiment and TFT elements, which are exemplary switching elements, connected to the organic light-emitting devices. In this FIGURE, two organic light-emitting devices and two TFT elements are illustrated. The detailed structure will be described below.

The display apparatus includes a substrate 1 composed of, for example, glass and a moisture-proof film 2 arranged thereon, the moisture-proof film 2 being configured to protect the TFT elements or organic compound layers. Reference numeral 3 denotes a metal gate electrode. Reference numeral 4 denotes a gate insulating film. Reference numeral 5 denotes a semiconductor layer.

TFT elements 8 each include the semiconductor layer 5, a drain electrode 6, and a source electrode 7. An insulating film 9 is arranged above the TFT elements 8. An anode 11 of each of the organic light-emitting devices is connected to a corresponding one of the source electrodes 7 through a contact hole 10. The structure of the display apparatus is not limited thereto. In each organic light-emitting device, one of the anode and the cathode may be connected to one of the source electrode and the drain electrode of a corresponding one of the TFT elements.

In this FIGURE, an organic compound layer 12 has a multilayer structure including a plurality of organic compound layers but is illustrated as if it had a single-layer structure, for convenience. A first protective layer 14 and a second protective layer 15 are arranged on cathodes 13 so as to suppress the degradation of the organic light-emitting devices.

The switching elements of the display apparatus according to this embodiment are not particularly limited. For example, a single-crystal silicon substrate, a metal-insulator-metal (MIM) element, and an amorphous silicon (a-Si) element may be easily used.

### EXAMPLES

### Example 1

### Synthesis of Exemplified Compound A3

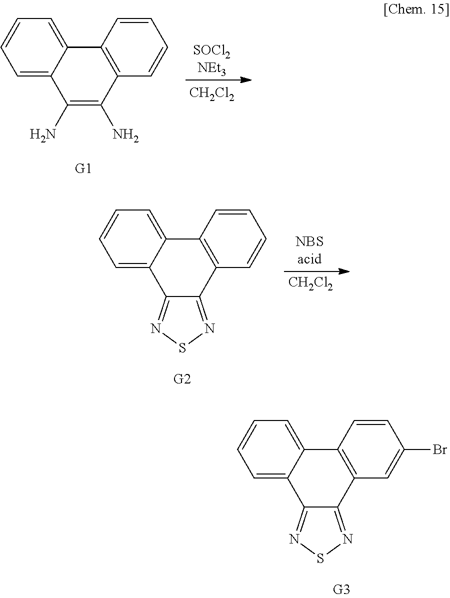

To 40 mL of a dichloromethane solution, 1.0 g (4.8 mmol) of G1, 1.9 g (19 mmol) of triethylamine, and 857 mg (7.2 mmol) of thionyl chloride were added. The mixture was heated to 50° C. and stirred for 6 hours. After cooling, water and chloroform were added thereto. The mixture was subjected to extraction with chloroform, followed by drying over sodium sulfate. The solvent was removed by evaporation. The residue was dissolved in toluene. The toluene solution was passed through silica gel. The solvent was removed by evaporation. Recrystallization of the residue from an ethyl acetate-toluene mixed solvent gave 0.30 g (yield: 33%) of G2 as pale-yellow needle crystals.

To a solution of 176 mg (0.745 mmol) of G2 and 22 mL of dichloromethane, 1.4 mL of trifluoromethanesulfonic acid was added. The mixture was stirred at room temperature for 30 minutes. Then water and chloroform were added thereto. After neutralization with sodium bicarbonate, the mixture was subjected to extraction with chloroform, followed by sodium sulfate. The solvent was removed by evaporation. The residue was purified by silica-gel column chromatography (mobile phase: 1:3 chloroform-heptane) to give 156 mg (43%) of G3 as a white solid.

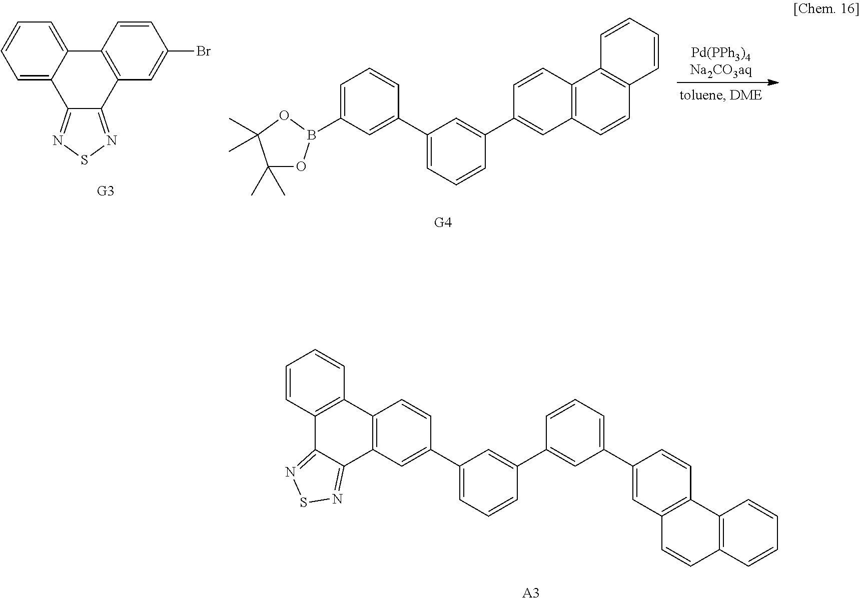 [SMILES: Brc1ccc2c3ccccc3c3nsnc3c2c1 ; CC1(C)OB(c2cccc(-c3cccc(-c4ccc5c(ccc6ccccc65)c4)c3)c2)OC1(C)C ; c1cc(-c2cccc(-c3ccc4c5ccccc5c5nsnc5c4c3)c2)cc(-c2ccc3c(ccc4ccccc43)c2)c1 ; "A3"]

To a mixture of 4 mL of toluene, 1 mL of DME, and 4 mL of an aqueous solution of 10 wt % sodium carbonate, 150 mg (0.476 mmol) of G3 and 239 mg (0.524 mmol) of G4 were added. Then 33 mg (0.029 mmol) of tetrakis(triphenylphosphine)palladium(0) was added thereto. The mixture was heated to 90° C. and stirred for 5 hours. After cooling, methanol and water were added thereto. The mixture was then filtered. The filtrate was purified by silica-gel chromatography (mobile phase: 1:2 chloroform-heptane) to give 235 mg (yield: 87%) of A3 as a white solid.

The M⁺ of exemplified compound A3, i.e., 565, was confirmed by mass spectrometry.

The structure of exemplified compound A3 was identified by ¹H NMR.

¹H NMR (CDCl₃, 500 MHz) δ (ppm): 8.83 (d, J=8.0 Hz, 1H), 8.78 (d, J=9.0 Hz, 2H), 8.76 (dd, J=8.0, 1.5 Hz, 1H), 8.72 (d, J=8.5 Hz, 1H), 8.64 (d, J=8.0 Hz, 1H), 8.18 (d, J=2.0 Hz, 1H), 8.08 (m, 2H), 8.02-7.99 (m, 2H), 7.91 (d, J=8.0 Hz, 1H), 7.83-7.71 (m, 12H)

The T1 level of exemplified compound A3 was measured in a dilute toluene solution and found to be 471 nm.

The T1 level was measured as follows: The toluene solution (1×10⁻⁴ mol/L) was cooled to 77 K. A phosphorescent component was measured at an excitation wavelength of 350 nm. The wavelength of a rising edge in the resulting spectrum was used as the T1 level. The measurement was performed with a spectrophotometer (Model: U-3010, manufactured by Hitachi, Ltd).

### Example 2

### Synthesis of Exemplified Compound A1

Exemplified compound A1 was synthesized as in Example 1, except that compound G5 described below was used in place of compound G4.

The M⁺ of exemplified compound A1, i.e., 541, was confirmed by mass spectrometry.

The T1 level of exemplified compound A1 was measured in a dilute toluene solution in the same way as in Example 1 and found to be 469 nm.

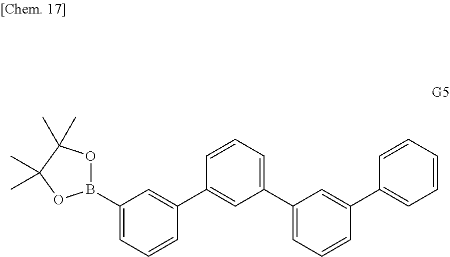 [SMILES: CC1(C)OB(c2cccc(-c3cccc(-c4cccc(-c5ccccc5)c4)c3)c2)OC1(C)C]

### Example 3

### Synthesis of Exemplified Compound A5

Exemplified compound A5 was synthesized as in Example 1, except that compound G6 described below was used in place of compound G4.

The M⁺ of exemplified compound A5, i.e., 581, was confirmed by mass spectrometry.

The T1 level of exemplified compound A5 was measured in a dilute toluene solution in the same way as in Example 1 and found to be 470 nm.

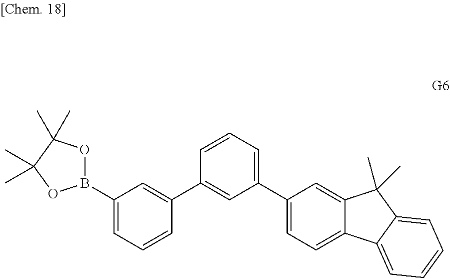 [SMILES: CC1(C)c2ccccc2-c2ccc(-c3cccc(-c4cccc(B5OC(C)(C)C(C)(C)O5)c4)c3)cc21]

### Example 4

### Synthesis of Exemplified Compound A7

Exemplified compound A7 was synthesized as in Example 1, except that compound G7 described below was used in place of compound G4.

The M⁺ of exemplified compound A7, i.e., 615, was confirmed by mass spectrometry.

The T1 level of exemplified compound A7 was measured in a dilute toluene solution in the same way as in Example 1 and found to be 470 nm.

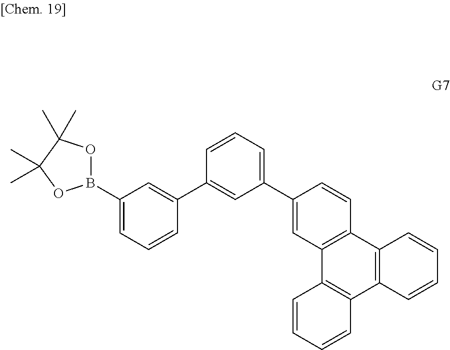 [SMILES: CC1(C)OB(c2cccc(-c3cccc(-c4ccc5c6ccccc6c6ccccc6c5c4)c3)c2)OC1(C)C]

### Example 5

### Synthesis of Exemplified Compound A8

Exemplified compound A8 was synthesized as in Example 1, except that compound G8 described below was used in place of compound G4.

The M⁺ of exemplified compound A8, i.e., 505, was confirmed by mass spectrometry.

The T1 level of exemplified compound A8 was measured in a dilute toluene solution in the same way as in Example 1 and found to be 469 nm.

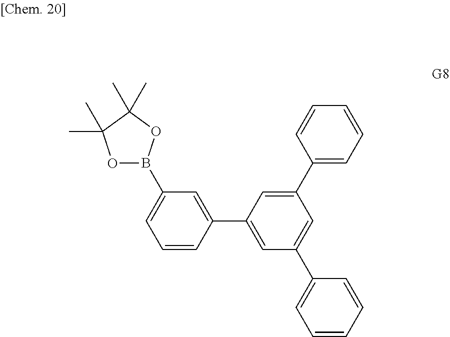 [SMILES: CC1(C)OB(c2cccc(-c3cc(-c4ccccc4)cc(-c4ccccc4)c3)c2)OC1(C)C]

### Example 6

### Synthesis of Exemplified Compound B3

Exemplified compound B3 was synthesized as in Example 1, except that compound G9 described below was used in place of compound G4.

The M⁺ of exemplified compound B3, i.e., 637, was confirmed by mass spectrometry.

The T1 level of exemplified compound B3 was measured in a dilute toluene solution in the same way as in Example 1 and found to be 469 nm.

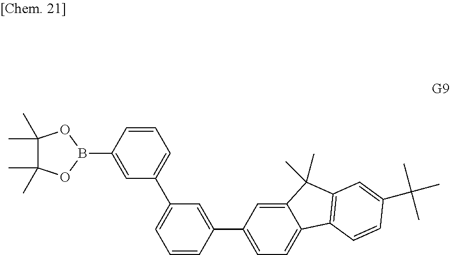 [SMILES: CC(C)(C)c1ccc2c(c1)C(C)(C)c1cc(-c3cccc(-c4cccc(B5OC(C)(C)C(C)(C)O5)c4)c3)ccc1-2]

### Example 7

### Synthesis of Exemplified Compound C4

Exemplified compound C4 was synthesized as in Example 1, except that compound G10 described below was used in place of compound G4.

The M⁺ of exemplified compound C4, i.e., 655, was confirmed by mass spectrometry.

The T1 level of exemplified compound C4 was measured in a dilute toluene solution in the same way as in Example 1 and found to be 471 nm.

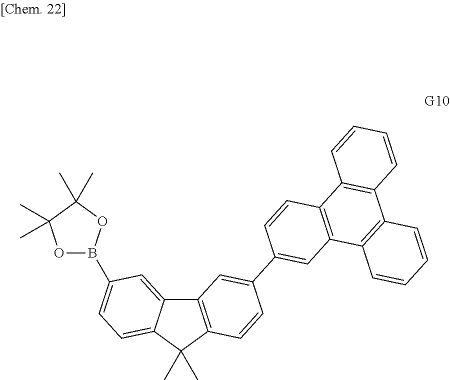 [SMILES: CC1(C)c2ccc(B3OC(C)(C)C(C)(C)O3)cc2-c2cc(-c3ccc4c5ccccc5c5ccccc5c4c3)ccc21]

### Example 8

In this example, an organic light-emitting device having a structure of anode/hole injection layer/hole transport layer/light-emitting layer/exciton-blocking layer/electron transport layer/electron injection layer/cathode arranged in that order on a substrate was produced by a method described below.

A transparent conductive supporting substrate (ITO substrate) produced by forming a 120-nm-thick ITO film serving as an anode by sputtering on a glass substrate was used. Organic layers and an electrode layer described below were continuously formed on the ITO substrate by vacuum evaporation using resistance heating in a vacuum chamber at 10⁻⁵ Pa in such a manner that the area of the facing electrodes was 3 mm².

Hole injection layer (165 nm): H1
Hole transport layer (10 nm): H2
Light-emitting layer (20 nm):

Host 1: H3,

Host 2: H4 (30% by weight),

Guest: F1 (10% by weight)

Exciton-blocking layer (10 nm): A3
Electron transport layer (10 nm): H5
Electron injection layer (20 nm): H6, Cs
Metal electrode layer (12.5 nm): Ag

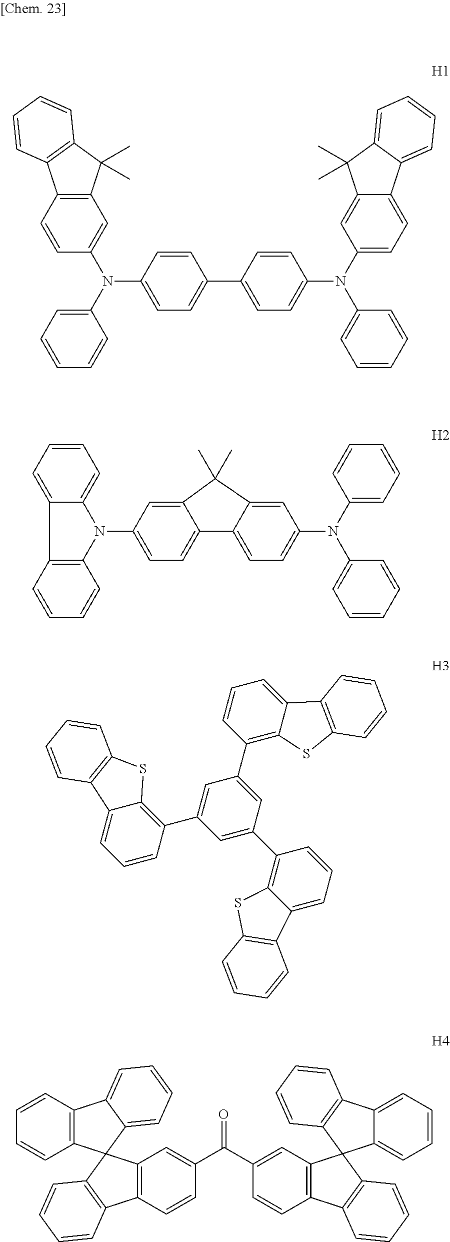 [SMILES: CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21 ; CC1(C)c2cc(N(c3ccccc3)c3ccccc3)ccc2-c2ccc(-n3c4ccccc4c4ccccc43)cc21 ; c1ccc2c(c1)sc1c(-c3cc(-c4cccc5c4sc4ccccc45)cc(-c4cccc5c4sc4ccccc45)c3)cccc12 ; O=C(c1ccc2c(c1)C1(c3ccccc3-c3ccccc31)c1ccccc1-2)c1ccc2c(c1)C1(c3ccccc3-c3ccccc31)c1ccccc1-2]

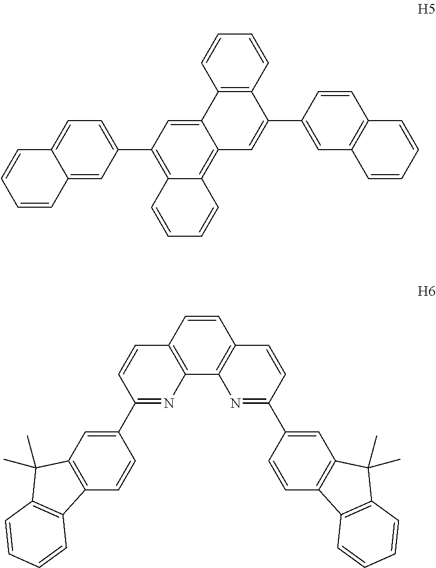

For the resulting organic light-emitting device, when a voltage of 4.5 V was applied between the ITO electrode serving as a positive electrode and the A1 electrode serving as a negative electrode, green light emission was observed at a luminous efficiency of 87 cd/A.

### RESULTS AND DISCUSSIONS

As described above, the organic compound according to aspects of the present invention has a high T1 level suitable for a green phosphorescent light-emitting device, high electron acceptability, and a deep LUMO level and is capable of forming a stable amorphous film. Thus, the organic light-emitting device including the organic compound according to aspects of the present invention can be driven at a low voltage and has high luminous efficiency.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-275135, filed Dec. 9, 2010, which is hereby incorporated by reference herein in its entirety.

### REFERENCE SIGNS LIST

8 TFT element
11 anode
12 organic compound layer
13 cathode

1. A phenanthrothiadiazole compound represented by one of general formulae [1] to [3]: 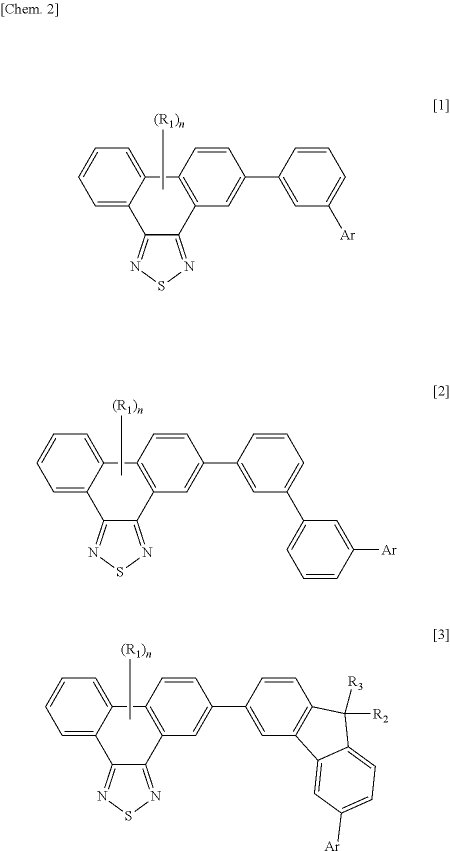
wherein in each of general formulae [1] to [3],
Ar represents a phenyl group, a phenanthryl group, a fluorenyl group, or a triphenylenyl group;
the substituent represented by Ar is unsubstituted or substituted with an alkyl group having 1 to 4 carbon atoms, a phenyl group, a phenanthryl group, a fluorenyl group, or a triphenylenyl group;
R₁ represents an alkyl group having 1 to 4 carbon atoms, n represents an integer of 0 to 3, and when n represents 2 or 3, alkyl groups represented by plural R₁'s may be the same or different; and
R₂ and R₃ each independently represent a hydrogen atom or an alkyl group having 1 to 4 carbon atoms.

2. An organic light-emitting device comprising:
a pair of electrodes; and
an organic compound layer arranged between the pair of electrodes,
wherein the organic compound layer comprises the phenanthrothiadiazole compound according to claim 1.

3. An organic light-emitting device comprising:
a pair of electrodes;
a light-emitting layer arranged between the pair of electrodes; and
an exciton-blocking layer in contact with the light-emitting layer,
wherein the exciton-blocking layer comprises the phenanthrothiadiazole compound according to claim 1.

4. The organic light-emitting device according to claim 3, wherein the light-emitting layer phosphoresces.

5. A display apparatus comprising:
a plurality of pixels,
wherein each of the plural pixels includes
the organic light-emitting device according to claim 2, and
a switching element connected to the organic light-emitting device.

6. An image output apparatus comprising:
an input unit configured to input image information; and
a display unit configured to output an image,
wherein the display unit includes a plurality of pixels, and
wherein each of the plural pixels includes
the organic light-emitting device according to claim 2, and
a switching element connected to the organic light-emitting device.

7. A display apparatus comprising:
a plurality of pixels,
wherein each of the plural pixels includes
the organic light-emitting device according to claim 3, and
a switching element connected to the organic light-emitting device.

8. An image output apparatus comprising:
an input unit configured to input image information; and
a display unit configured to output an image,
wherein the display unit includes a plurality of pixels, and
wherein each of the plural pixels includes
the organic light-emitting device according to claim 3, and
a switching element connected to the organic light-emitting device.

9. A display apparatus comprising:
a plurality of pixels,
wherein each of the plural pixels includes
the organic light-emitting device according to claim 4, and
a switching element connected to the organic light-emitting device.

10. An image output apparatus comprising:
an input unit configured to input image information; and
a display unit configured to output an image,
wherein the display unit includes a plurality of pixels, and
wherein each of the plural pixels includes
the organic light-emitting device according to claim 4, and
a switching element connected to the organic light-emitting device.

11. An illuminating apparatus comprising the organic light emitting device according to claim 2.

12. An illuminating apparatus comprising the organic light emitting device according to claim 3.

13. An illuminating apparatus comprising the organic light emitting device according to claim 4.
